(19) United States
(12) Patent Application Publication
(10) Pub. No.: US 2018/0116542 A1
(43) Pub. Date: May 3, 2018

(54) BRAIN ELECTRODE APPARATUS, METHOD FOR MEASURING SIGNAL OUTPUTTED FROM BRAIN NERVOUS TISSUE, AND METHOD FOR APPLYING SIGNAL TO BRAIN NERVOUS TISSUE

(71) Applicant: PROMOVEO LLC, Chuo-ku Tokyo (JP)

(72) Inventor: Shigeru Ogino, Tokyo (JP)

(21) Appl. No.: 15/350,176

(22) Filed: Nov. 14, 2016

Related U.S. Application Data
(63) Continuation of application No. PCT/JP2016/082157, filed on Oct. 28, 2016.

(51) Int. Cl. A61B 5/0478; A61N 1/05

(52) U.S. Cl. CPC A61B 5/0478; A61N 1/0534

(57) ABSTRACT

A brain electrode apparatus is a brain electrode apparatus including an electrode that is capable of being inserted into a brain, and the brain electrode apparatus includes a long-shaped probe, an electrode part formed on the probe, and an inserting member that is detachably inserted in a first hole formed on a plane face of the probe having flexibility. Further, the probe includes a first area on the electrode part side with respect to the position where the first hole is formed, and a second area on the opposite side of the electrode part side with respect to the position where the first hole is formed, and the inserting member is inserted into the first hole in a state where the probe is bent at the position of the first hole such that the second area overlaps the first area.

### CROSS-REFERENCE TO RELATED APPLICATIONS

The present application is a continuation application of International Application number PCT/JP2016/082157, filed on Oct. 28, 2016. The content of this application is incorporated herein by reference in their entirety.

### BACKGROUND OF THE INVENTION

Conventionally, elucidation of brain function is required for a medical treatment and the like. Therefore, faint electric signals (action potentials of cells) flowing in brain nervous tissue is measured by inserting an electrode (hereinafter, a brain electrode) into a brain.

As a brain electrode, Japanese Unexamined Patent Application Publication No. 2009-285153 discloses, for example, a nerve electrode in which a plurality of electrode wirings, which include an electrode part and a wiring part, is provided on an insulating layer that consists of a flexible insulating material. This nerve electrode in an inserted state is used for applying electrical stimulation to a brain and for measuring electrical activity of the brain.

By the way, a brain electrode is desirably inserted into a deep part of a brain for precisely elucidating various activities of the brain. Accordingly, a means in which an inserting member such as a needle is used to insert an electrode into the deep part of the brain has been considered.

However, the brain electrode disclosed in Patent Document 1 has difficulty in inserting the nerve electrode into the deep part of the brain by using the inserting member because the nerve electrode is formed like a planar plate. On the other hand, when the brain electrode is made with a complicated structure for being inserted into the brain by the inserting member, there is a possibility of damaging the brain nervous tissue when the brain electrode is inserted.

### BRIEF SUMMARY OF THE INVENTION

This invention focuses on these points, and an object of the invention is to provide a brain electrode apparatus that is capable of reducing influences on brain tissue when a brain electrode is inserted in a brain.

In the first aspect of the present invention, a brain electrode apparatus including an electrode that is capable of being inserted into a brain, the brain electrode apparatus comprising a long-shaped probe, an electrode part that is formed on the probe, and an inserting member that is detachably inserted in a first hole formed on a planar face of the probe, the probe being flexible is provided.

In the second aspect of the present invention, a method for measuring a signal outputted from brain nervous tissue, comprising inserting a long-shaped probe of a brain electrode apparatus into a brain, the brain electrode apparatus including the long-shaped probe, an electrode part that is formed on the probe, and an inserting member that is detachably inserted in a first hole formed on a plane face of the flexible probe, pulling out the inserting member in a state where the probe is inserted in a brain, and obtaining a signal outputted through the electrode part in a state where the probe is inserted in the brain is provided.

In the third aspect of the present invention, a method for applying a signal to brain nervous tissue comprising inserting a long-shaped probe of a brain electrode apparatus into a brain, the brain electrode apparatus including the long-shaped probe, an electrode part that is formed on the probe, and an inserting member that is detachably inserted in a first hole formed on a plane face of the flexible probe, pulling out the inserting member in a state where the probe is inserted in a brain, and applying a stimulating signal to the brain nervous tissue through the electrode part in a state where the probe is inserted in the brain is provided.

### BRIEF DESCRIPTION OF THE DRAWINGS

FIG. 1A shows an appearance of a brain electrode apparatus S according to the present exemplary embodiment.

FIG. 1B shows an appearance of a brain electrode apparatus S according to the present exemplary embodiment.

FIG. 2 shows a configuration of the brain electrode apparatus S according to the present exemplary embodiment.

FIG. 3 shows members of the brain electrode apparatus S in a separated state according to the present exemplary embodiment.

FIG. 4 shows an enlarged view of a probe part 10.

FIG. 5 shows an enlarged view of a vicinity of a first hole 103 in the probe 100.

FIG. 6 shows a perspective view of the probe 100 to be inserted into a brain.

FIG. 7 shows a perspective view of an example of a variation of the probe 100 to be inserted into a brain.

### DETAILED DESCRIPTION OF THE INVENTION

Hereinafter, the present invention is described though the exemplary embodiment of the invention but the undermentioned embodiment does not limit the invention according to the claims and all of the combinations of characteristics described in the embodiment are not necessarily essential for a solution of the invention.

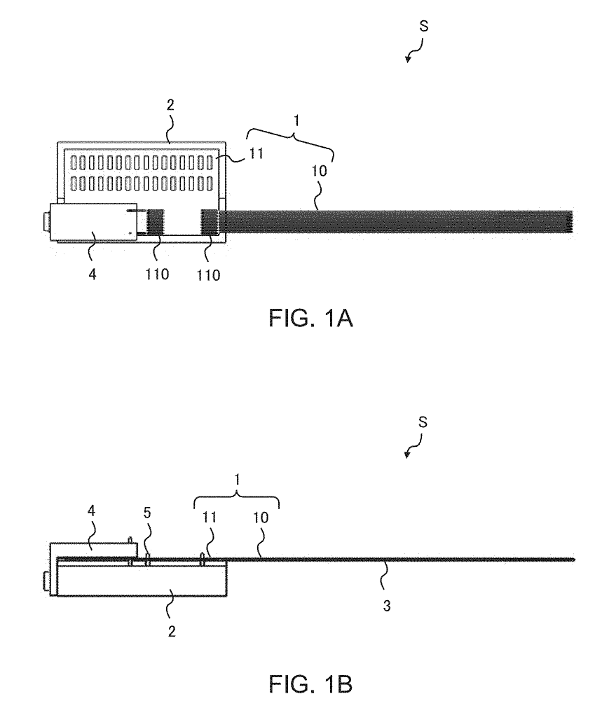

FIG. 1 shows an appearance of a brain electrode apparatus S according to the present exemplary embodiment. FIG. 1A shows a schematic planar view of a configuration of the brain electrode apparatus S according to the present exemplary embodiment. FIG. 1B shows a schematic side view of the configuration of the brain electrode apparatus S according to the present exemplary embodiment.

The brain electrode apparatus S is an apparatus that includes an electrode capable of being inserted into a brain, and is an apparatus for obtaining an electric signal outputted from the brain tissue through the electrode and for applying the electric signal to the brain tissue. The electric signal obtained by the brain electrode apparatus S is transmitted to an analyzing device such as a computer through a connector (not shown in figures), and various activities of the brain can be elucidated by using the analyzing device.

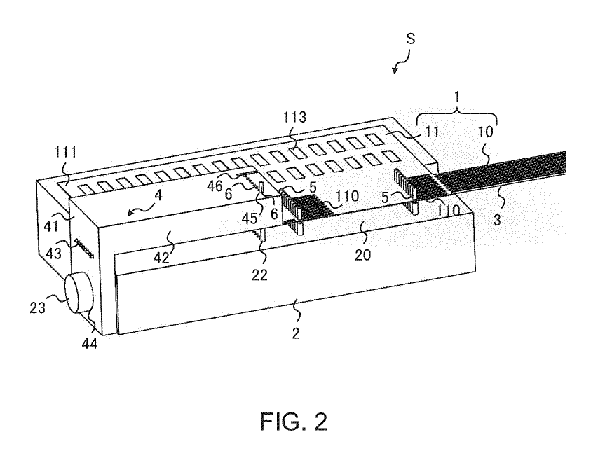

When a user pushes a brain electrode apparatus S towards the brain, a probe part 10, which is provided with an electrode, is inserted into the brain by an inserting member 3 that is explained below with reference to FIG. 2. The electrode of the brain electrode apparatus S is inserted to a depth of, for example, 10 to 100 (mm), but may be inserted to a depth of 100 (mm) or more. The brain electrode apparatus S obtains the electric signal showing the activity of the brain nervous tissue and transmits it to the analyzing device when the electrode is inserted in the brain.

The brain electrode apparatus S includes a flexible substrate 1, a base 2, an inserting member 3, an inserting member fixing part 4, and a pin 5. The flexible substrate 1, for example, has a laminated structure made of polyimide resin. The flexible substrate 1 includes a probe part 10 and a flat part 11.

The probe part 10 has a long shape. The probe part 10 has a thickness of a few μm to several tens of μm and has flexibility. The probe part 10 that is inserted into the brain can be bent by following movements of soft brain nervous tissue because the probe part 10 has flexibility. Consequently, by the whole probe part 10 moving inside of the brain, the electrode can be prevented from being relatively displaced with respect to the brain nervous tissue and the probe part 10 can be prevented from being snapped.

The flat part 11 is a rectangular-shaped part in which the probe 10 protrudes from a side of the flat part 11. An interface part (for example, a land) that is provided with a connector for connecting with the analyzing device is formed on the flat part 11.

The base 2 is a member that is fixed to the flexible substrate 1. The base 2 is formed with a hole for detachably fixing the pin 5. The inserting member 3 is a stick-shaped member and an end of the inserting member 3 is thin like a needle. The inserting member 3 is made of, for example, resin or metal. The inserting member 3 may be an optical fiber or a thin tube.

The inserting member fixing part 4 is a member for fixing the position of the inserting member 3 in the longitudinal direction, and is detachable to the flat part 11 and the base 2. The inserting member fixing part 4 shown in FIG. 1B has an L-shape, but the shape of the inserting member fixing part 4 is arbitrary.

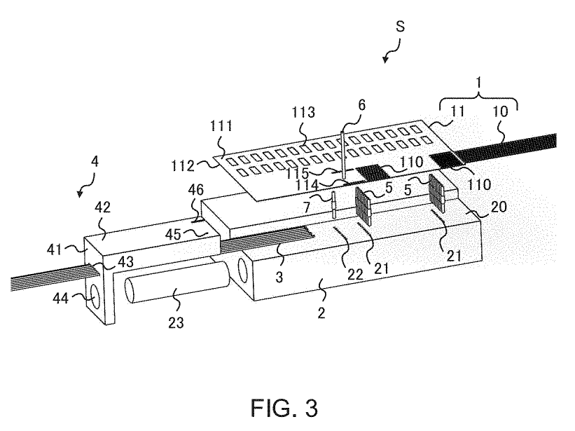

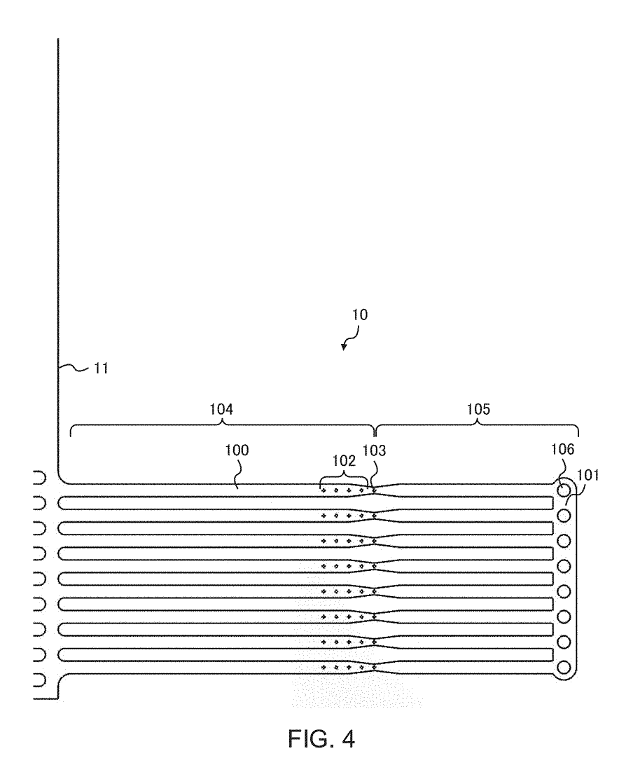

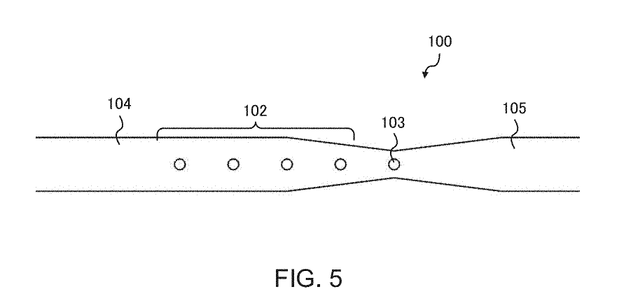

FIG. 2 shows a configuration of the brain electrode apparatus S according to the present exemplary embodiment. FIG. 3 shows members, in a separated state, that configure the brain electrode apparatus S according to the present exemplary embodiment. FIG. 4 shows an enlarged view of a probe part 10. FIG. 5 shows an enlarged view of a vicinity of a first hole 103 in the probe 100. Hereinafter, details of the brain electrode apparatus S are explained with reference to FIGS. 2 to 5.

The probe part 10 includes a plurality of probes 100 as shown in FIG. 4. Each of the plurality of probes 100 has a connecting part 101, an electrode part 102, the first hole 103, and a second hole 106. The probe 100 has a long shape and includes a first area 104 and a second area 105. The first area 104 is an area on the electrode part 102 side with respect to the position where the first hole 103 is formed. The second area 105 is an area on the opposite side (that is, on the end side) of the electrode part 102 side with respect to the position where the first hole 103 is formed.

The probe 100 is connected to the flat part 11 and is formed by protruding from the flat part 11. In FIG. 4, the probe part 10 includes eight probes 100, but the number of the probes 100 is arbitrary.

The connecting part 101 connects the plurality of probes 100 at the end of the probe part 10. The connecting part 101 is provided to enable easy handling of the flexible substrate 1 when the probe part 10 is manufactured. When the connecting part 101 is provided, for example, it is possible to reduce the time for inserting the inserting member 3 into a hole provided to the probe 100 after the probe 100 is bent.

The electrode part 102 is formed on a planar face of the probe 100. The electrode part 102 includes a plurality of electrodes. The plurality of electrodes are arranged in a straight line in the longitudinal direction of the probe 100. FIG. 4 shows the electrode part 102 that includes four electrodes, but the number of electrodes included in the electrode part 102 is arbitrary. The electrode part 102 is exposed to at least one side of both sides of the probe 100 to contact with the brain nervous tissue. The electrode part 102 contacts the brain nervous tissue in a state where the electrode included in the brain electrode apparatus S is inserted in the brain and detects the electric signal (for example, a variation of the potential) generated by the brain nervous tissue.

The electrode part 102 is connected to the land formed on the flat part 11 through a metallic pattern formed in the probe 100. It should be noted that the electrode part 102 and a metallic pattern consist of a material containing at least one of, for example, gold, platinum, and iridium.

The first hole 103 is a hole into which the inserting member 3 is inserted. The first hole 103 is formed on the planar face of the flexible probe 100. As shown in FIG. 5, the width of the probe 100 at a position where the first hole 103 is formed narrower than the width of the probe 100 at a position where the electrode part 102 is formed. The probe 100 can be easily bent at the first hole 103 by having such a configuration. Further, invasiveness of the probe 100 to the brain can be reduced by having such a configuration.

The second hole 106 is a hole into which the inserting member 3 is inserted. The second hole 106 is formed at the end of the second area 105. An inside diameter of the second hole 106 is larger than an inside diameter of the first hole 103.

Next, the flat part 11 is explained. As shown in FIG. 2 and FIG. 3, the flat part 11 includes a plurality of third holes 110, a plurality of lands 113, a hole 114, and a hole 115. The third hole 110 is a hole into which the pin 5 is inserted and is formed with an elliptical shape. In the present exemplary embodiment, there are eight probes 100, and two lines each including nine of the third holes 110 are formed on the flat part 11. A first face 111 of the flat part 11 is a face that is opposite to the base 2 side face of the flat part 11, and the first face 111 is a face on the same side of the face where the electrode part 102 of the probe 100 is provided. A second face 112 is the base 2 side face of the flat part 11.

The pin 5 is a member for fixing the flexible substrate 1 to the base 2. Further, the pin 5 has a function for guiding the inserting member 3 and positioning the inserting member 3 when the inserting member 3 is moved. As shown in FIG. 2 and FIG. 3, when the probe part 10 includes eight probes 100, two lines of nine pins 5 are inserted in two lines of nine holes 21.

The pin 5 is, for example, a stepped pin including a plurality of cylindrical end parts and middle parts. Outside diameters of the plurality of end parts are smaller than the outside diameters of the middle parts. A first end part of the pin 5 is inserted in the hole 21 formed on the base 2. A second end part of the pin 5 passes through the third hole 110 of the flexible substrate 1. The flexible substrate 1 is supported by a step between the second end part and the middle part of the pin 5. The outside diameter of the middle part of the pin 5 is set to make a space between adjacent pins 5 larger than the outside diameter of the inserting member 3. It should be noted that the pin 5 does not have to be a stepped pin.

The lands 113 are connected to one or more metallic patterns drawn from one or more electrodes included in the electrode part 102 on the first face 111 of the flat part 11. The electric signals detected by the electrode part 102 are outputted to the lands 113 through the metallic pattern. A connector for connecting with an external device such as an analyzing device is provided on the lands 113, and the electric signal detected by the electrode part 102 is transmitted to the analyzing device through the lands 113 and the connector.

The hole 114 is a hole into which the pin 6 is inserted. The pin 6 is a member for fixing the inserting member fixing part 4 to the flexible substrate 1 and the base 2. The pin 6 has, for example, a cylindrical shape. The hole 114 is provided on the flat part 11, and the user can fix the inserting member fixing part 4 to the flexible substrate 1 and the base 2 by inserting the pin 6 into the hole 114. The hole 115 is a hole into which a pin 7 is inserted.

As shown in FIG. 2 and FIG. 3, the base 2 includes a concave part 20, a hole 21, a hole 22, and a convex part 23. The concave part 20 is an area that is provide on the face on the side where the probe 10 is provided in the base 2, and is made lower than the face on the side where the probe 10 is not provided in the base. The concave part 20 forms a space for the inserting member 3 to be arranged.

The hole 21 is a hole in which the pin 5 is inserted. The plurality of holes 21 are provided on a bottom face of the concave part 20. The plurality of holes 22 are provided on the bottom face of the concave part 20, and the pin 6 and the pin 7 are inserted in these holes.

The convex part 23 is provided on the base 2 and protrudes from the base 2 towards the inserting member fixing part 4. The convex part 23 is used for positioning the inserting member fixing part 4. Specifically, the convex part 23 has a function that guides a lateral position when the inserting member fixing part 4 is moved towards the base 2. The convex part 23 may be detachably provided to the base 2. For example, the convex part 23 is a cylindrical member and may have a configuration for being used by being inserted into a hole provided on the base 2.

Each of the plurality (eight in the present exemplary embodiment) of inserting members 3 is detachably inserted in the first hole 103 formed on the plane face of the flexible probe 100 on the second face 112 side that is opposite to the first face 111 of the flat part 11. The outside diameter of the inserting member 3 is smaller than the inside diameter of the second hole 106. Further, an end part of the inserting member 3 is formed in a needle-like shape to be easily inserted into the first hole 103 and the end part has, for example, a circular cone shape. Because the inserting member 3 has such a shape, the inserting member 3 can pass through the second hole 106 at any position, and only a portion of the end can pass through the first hole 103.

The plurality of the third holes 110 are formed at positions on both sides of the inserting member 3 in a short direction of the inserting member 3. Because the brain electrode apparatus S has such a configuration, the user can move the inserting member 3 in a proper direction by inserting the inserting member 3 between the plurality of the pins 5 that are inserted in the plurality of the third holes 110 when the inserting member 3 is moved. Further, because the inserting member 3 is arranged at the second face 112 side in the brain electrode apparatus S, the inserting member 3 can be prevented from being interfered by a cable and connector that are provided on the lands 113 formed on the first face 111.

The inserting member fixing part 4 is an L-shaped member that includes a vertical plate part 41 and a horizontal plate part 42. A hole 43 into which the inserting member 3 is inserted is formed in the vertical plate part 41. The vicinity of the rear end of the inserting member 3 is fixed to the inserting member fixing part 4 when the inserting member 3 is inserted into the hole 43. Further, a hole 44 is formed in the vertical plate part. The hole 44 is a positioning hole through which the convex part 23 that is provided on the base 2 passes.

The direction of the horizontal plate part 42 is orthogonal to the direction of the vertical plate part 41. A hole 45 and a groove 46 are formed in the horizontal plate part 42. The hole 45 is a hole into which the pin 6 is inserted. The pin 6 passes through the hole 45 of the inserting member fixing part 4 and the hole 114 of the plate part 11, and is inserted into the hole 22 of the base 2. The groove 46 is formed with an elongated shape in a moving direction of the inserting member fixing part 4, and the pin 7 that is inserted into the hole 22 and the hole 115 enters the groove 46 as the inserting member fixing part 4 is moved to the end side of the probe part 10. The inserting member 3 can be stably moved in an inserting direction due to the groove 46 formed on the horizontal plate part 42.

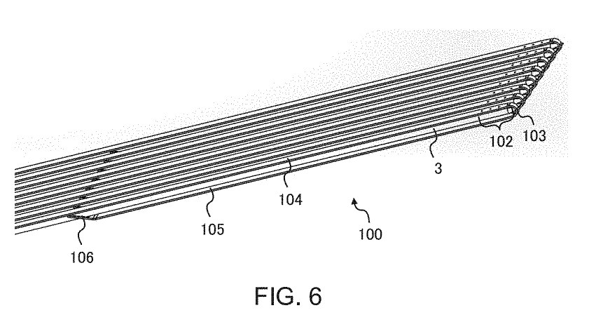

FIG. 6 shows a perspective view of the probe 100 to be inserted into a brain. FIG. 6 shows the inserting member 3 being inserted in the second hole 106 and the first hole 103 that are formed on the probe 100 with the end of the probe 100 being bent. The inserting member 3 is inserted in the first hole 103 by passing through the second hole 106 in a state where the probe 100 is bent at the position of the first hole 103 such that the second area 105 overlaps the first area 104.

In such a manner, a bending part with a predetermined bending curvature is formed at the end of the probe 100 by bending the probe 100. By bending the probe 100 at the position of the first hole 103, the inserting member 3 can be prevented from being separated from the first hole 103 due to the impact generated when the brain electrode apparatus S is carried and the probe part 10 is inserted into the brain. Further, the end part of the probe 100 is bent when the probe part 10 is inserted, and the bending curvature of the bending part as well as the space between the first area 104 and the second area 105 gets smaller. Consequently, the probe 100 can reduce invasiveness to the brain when being inserted. It should be noted that a portion of the second area 105 may be cut and removed when the probe 100 is inserted in the brain.

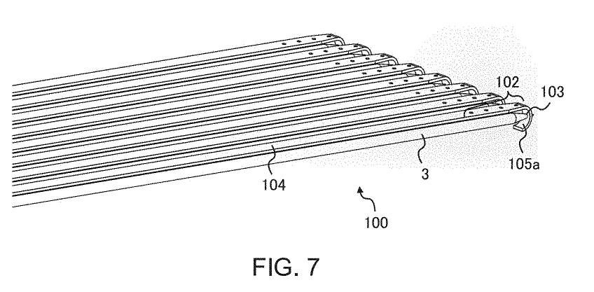

FIG. 7 shows a perspective view of an example of a variation of the probe 100 to be inserted into a brain. In the variation shown in FIG. 7, the second area 105a is shorter than the second area 105, and the end of the second area 105a does not have the second hole. Also, the tip part of the first hole 103 is bent in a J-shape. Because the probe 100 has such a configuration, the probe 100 can reduce the invasiveness to the brain compared with the probe part 10 including the second hole 106 and the second area 105.

### [Method for Manufacturing the Brain Electrode Apparatus S According to the Present Exemplary Embodiment]

A method for manufacturing the brain electrode apparatus S according to the present exemplary embodiment is explained with reference to the FIG. 3. First, the plane-shaped flexible substrate 1 including the electrode part 102 is formed. Specifically, the flexible substrate 1 is formed by forming the second layer to cover the metallic pattern after the electrode part 102 and the metallic pattern are formed on a first layer. It should be noted that the first layer and the second layer consist of, for example, polyimide, and the electrode part 102, and the metallic patterns consist of a material containing at least one of, for example, gold, platinum, and iridium.

Next, the probe part 10 is bent at the position of the first hole 103 such that the second area 105 overlaps the first area 104 in the probe 100 of the flexible substrate 1. A space between the first hole 103 and the second hole 106 in the second area 105 is cut as necessary. And then, the flexible substrate 1 is fixed to the base 2 by using the pin 5.

Next, the inserting member 3 is inserted into the hole that is formed in the inserting member fixing part 4. And then, by moving the inserting member fixing part 4, the inserting member 3 that is fixed to the inserting member fixing part 4 is moved towards the end of the probe 100 along the positions between the plurality of pins 5.

Next, the end of the inserting member 3 is inserted into the second hole 106 and the first hole 103. On this occasion, rubber and gel are attached to the end of the inserting member 3 that protrudes from the first hole 103 to prevent the inserting member 3 from being released from the hole. The rubber and the gel are removed before the inserting member 3 is inserted into the brain. And then, the inserting member fixing part 4 is fixed to the base 2 by inserting the pin 6 to the hole 45 formed to the inserting member fixing part 4. Lastly, the inserting member 3 is fixed to the inserting member fixing part 4 by applying an adhesive to the position where the inserting member 3 and the inserting member fixing part 4 contact each other. In this manner, the brain electrode apparatus S is manufactured.

[Method for Measuring Signals Outputted from Brain Nervous Tissue]

Next, a method for measuring the signals outputted from the brain nervous tissue according to the present exemplary embodiment is explained. First, the probe 100 is inserted into the brain by moving the brain electrode apparatus S towards the brain. For example, the probe 100 is inserted until the electrode part 102 that is provided to the probe 100 reaches a portion of the brain nervous tissue to be measured. Then, the inserting member 3 is pulled out in a state where the probe 100 is inserted in the brain. After the inserting member 3 is pulled out, the brain electrode apparatus S is connected to an analyzing device (for example, a computer) through a connector and a cable that are connected to the lands 113. Subsequently, the signal outputted through the electrode part 102 is obtained by operating the analyzing device. In this manner, the signal outputted from the brain nervous tissue can be measured.

### [Method for Applying Signal to Brain Nervous Tissue]

Next, a method for applying the signal to the brain nervous tissue is explained. First, the probe 100 of the brain electrode apparatus S including the long-shaped probe 100, the electrode part 102 that is formed on the probe 100, and the inserting member 3 that is detachably inserted in the first hole 103 formed on the planar face of the flexible probe 100 is inserted into the brain. Next, the inserting member 3 is pulled out in a state where the probe 100 is inserted in the brain. Then, a stimulating signal is applied to the brain nervous tissue through the electrode part 102 in a state where the probe 100 is inserted in the brain. In this manner, the stimulating signal can be applied to the brain nervous tissue.

### [Effects Realized by Brain Electrode Apparatus S According to Present Exemplary Embodiment]

The brain electrode apparatus S according to the present exemplary embodiment is a brain electrode apparatus S that includes an electrode capable of being detachably inserted in the brain, and the brain electrode apparatus S includes the long-shaped probe 100, the electrode part 102 that is formed on the probe 100, and the inserting member 3 that is detachably inserted in the first hole 103 formed on the planar face of the flexible probe 100. The brain electrode apparatus S according to the present exemplary embodiment can reduce influences on the brain tissue when the brain electrode is inserted into the brain by having such a configuration.

Further, because the brain electrode apparatus S includes the probe 100 that uses a polymer such as polyimide as a forming material, a brain electrode that is easy to insert into a deep part of the brain, and the operation of which is highly stable when the electric signals are measured and the stimulating signals are applied, can be provided at a low cost. Further, because the probe 100 contains a polymer as the forming material, the end portion can be easily bent.

The present invention is explained on the basis of the exemplary embodiments of the present disclosure. New exemplary embodiments generated by arbitrary combinations of them are included in the exemplary embodiments of the present invention. Effects of the new exemplary embodiments brought by the combinations also have the effects of the original exemplary embodiments. Further, the technical scope of the present invention is not limited to the scope explained in the above embodiments and it is possible to make various changes and modifications within the scope of the invention.

In the above explanation, the first hole 103, the second hole 106, the hole 114, and the hole 115 are perfect circles, but the shapes of these holes are not limited to perfect circles and they may be ellipses or may have shapes with varying curvatures. Further, the third hole 110 is formed in an elliptical shape in the above but, the shape of the third hole 110 may be other shapes such as a rectangle.

Further, an example of the probe part 10 including eight probes 100 is illustrated in the above, but the number of the probes 100 is arbitrary. The number of inserting members 3, the number of pins 5, and the number of holes formed for each part are determined according to the number of probes 100.

What is claimed is:

1. A brain electrode apparatus including an electrode that is capable of being inserted into a brain, the brain electrode apparatus comprising:
a long-shaped probe;
an electrode part that is formed on the probe; and
an inserting member that is detachably inserted in a first hole formed on a planar face of the probe, the probe being flexible.

2. The brain electrode apparatus according to claim 1, wherein
the probe includes a first area on the electrode part side with respect to a position where the first hole is formed and a second area on the opposite side of the electrode part side with respect to the position where the first hole is formed, and
the inserting member is inserted into the first hole in a state where the probe is bent at the position of the first hole such that the second area overlaps the first area.

3. The brain electrode apparatus according to claim 2, wherein
a second hole is formed in an end portion of the second area, and
the inserting member passes through the second hole and is inserted in the first hole in a state where the probe is bent.

4. The brain electrode apparatus according to claim 1, wherein
width of the probe at the position where the first hole is formed narrower than width of the probe at the position where the electrode part is formed.

5. The brain electrode apparatus according to claim 1, further comprising:
a flat part in which a plurality of third holes are formed for pins for positioning the inserting member to be inserted, the flat part being connected with the probe.

6. The brain electrode apparatus according to claim 5, wherein
the plurality of third holes are formed at the positions on both sides of the inserting member in a short direction of the inserting member.

7. The brain electrode apparatus according to claim 6, further comprising:
an inserting member fixing part that fixes the position of the inserting member in the longitudinal direction, the inserting member fixing part being detachable from the flat part and the base in which a hole is formed for detachably fixing the pin.

8. The brain electrode apparatus according to claim 7, wherein
the base includes a convex part for positioning the inserting member fixing part, and
a positioning hole that passes through the convex part is formed on the inserting member fixing part.

9. The brain electrode apparatus according to claim 5, wherein
one or more lands that are connected to one or more metallic patterns that are drawn from one or more electrodes included in the electrode part is formed on a first face of the flat part, and
the inserting member is arranged at a second face side that is opposite to the first face of the flat part.

10. The brain electrode apparatus according to claim 9, wherein
the first face of the flat part is the face on the same side as the face where the electrode part of the probe is provided.

11. A method for measuring a signal outputted from brain nervous tissue, comprising:
inserting a long-shaped probe of a brain electrode apparatus into a brain, the brain electrode apparatus including the long-shaped probe, an electrode part that is formed on the probe, and an inserting member that is detachably inserted in a first hole formed on a plane face of the flexible probe,
pulling out the inserting member in a state where the probe is inserted in a brain, and
obtaining a signal outputted through the electrode part in a state where the probe is inserted in the brain.

12. A method for applying a signal to brain nervous tissue comprising:
inserting a long-shaped probe of a brain electrode apparatus into a brain, the brain electrode apparatus including the long-shaped probe, an electrode part that is formed on the probe, and an inserting member that is detachably inserted in a first hole formed on a plane face of the flexible probe,
pulling out the inserting member in a state where the probe is inserted in a brain, and
applying a stimulating signal to the brain nervous tissue through the electrode part in a state where the probe is inserted in the brain.
